(19) United States
(12) Patent Application Publication
(10) Pub. No.: US 2009/0208568 A1
(43) Pub. Date: Aug. 20, 2009

(54) Gellan Seamless Breakable Capsule and Process for Manufacturing Thereof

(75) Inventors: Jean-Michel Hannetel, Grasse (FR); Didier Hartmann, Cannes La Bocca (FR); Nathalie Coursieres, Saint Cezaire Sur Siagne (FR); Jean Mane, Grasse (FR)

Correspondence Address: YOUNG & THOMPSON, 209 Madison Street, Suite 500, ALEXANDRIA, VA, 22314, US

(73) Assignee: V MANE FILS, BAR SUR LOUP (FR)

(21) Appl. No.: 11/922,574

(22) PCT Filed: Jun. 21, 2006
(86) PCT No.: PCT/IB2006/002905

§ 371 Date: Feb. 1, 2008

(30) Foreign Application Priority Data
Jun. 21, 2005 (EP) PCT/EP05/08502
Aug. 5, 2005 (EP) PCT/EP05/09226

(51) Int. Cl. A61K 9/48; B32B 5/16; B05D 3/00; A61K 9/28; A61P 43/00; A61J 3/07; A61Q 11/00; A61L 9/04; A61Q 13/00; A23L 1/48

(52) U.S. Cl. 424/452; 428/403; 427/384; 427/2.14; 264/4; 424/49; 512/4; 426/89

(57) ABSTRACT

The invention relates to a process for manufacturing a seamless breakable capsule, comprising—co-extruding an external and hydrophilic liquid phase, and an internal and lipophilic liquid phase, in order to form a capsule constituted of a core comprising the internal and lipophilic phase, and a shell comprising the external and hydrophilic phase,—immersing into an aqueous solution containing a curing agent, wherein the external liquid phase includes a gelling agent comprising gellan gum alone or in combination with another gelling agent, a filler, and a divalent metal sequestering agent, and to breakable capsules comprising a core and a shell, wherein the shell includes a gelling agent comprising gellan gum alone or in combination with another gelling agent, a filler, and a divalent metal sequestering agent.

The present invention relates to a breakable capsule having a fluid core and a solid or fluid breakable shell.

In this invention, the term “capsule” means a spherical or substantially spherical delivery system of a substance, said substance being hereinafter referred to as “the core”, and said substance being encapsulated into a shell, the shell being breakable and releasing the core when broken or ruptured. The term fluid means flowing as opposed to being in a solid state. According to the invention, the term fluid includes finely divided solids, such as a powder, and also gel, or any physical state of a product wherein said product changes shape or direction uniformly, in response to an external force imposed upon it. According to the invention, fluid preferably refers to a flowable or gellified product.

The term “breakable capsule” refers to a capsule as hereabove defined, wherein the shell can be ruptured by means of a pressure, which results in the release of the core. According to an embodiment, the capsule of the invention may be specifically designed to be incorporated into a fluid medium such as for example a gel, a pasty or a liquid medium containing water; in this embodiment, the capsules may be suspended or mixed by any suitable means in order to bring an visual effect of homogeneous dispersion of the capsules in the medium; advantageously, the shell and/or the core of the capsule is coloured. According to another embodiment, the capsule of the invention is dispersed into a solid or fluid medium, such as for example a powder; advantageously, the shell and/or the core of the capsule is coloured.

Such capsules are useful for numerous applications, such as in oral care application (toothpaste, mouthwash, gums . . . ), in food applications such as confectionary, dairy, bakery, savory, in neutraceutical applications or in pharmaceutical or in personal care products such as cosmetic products and the like.

In the present patent application, the term “capsule” will be used to designate any size of capsules, including macrocapsules and microcapsules and preferably capsule which larger diameter is from 0.5 mm up to 8 mm, preferably 1 to 5 mm; more preferably 1.2 to 3 mm.

It is of particular interest to obtain seamless capsules, as the breakability of a welded capsule (also designated in the prior art as softgel or hard capsule) may be influenced by the easy or unwanted rupture of the weld.

Fuji patent application JP10291928 describes a capsule obtained through a co-extrusion process, wherein the external liquid phase comprises gellan and calcium salts. Gellan gum, first discovered in 1978, is produced by the microorganism Sphingomonas elodea.

The Applicant has found that the production of gellan capsule through the Fuji process was not satisfactory and resulted in poor quality capsules and in processing difficulties, because the gellan was actually gelling during the co-extrusion, and it was not possible to obtain spherical and homogeneous breakable capsules.

For this reason, the Applicant tried to improve the Fuji process and found that the drawbacks of the prior art process may be due to the presence of calcium salts, and more generally to divalent metal salts in gellan during the co-extrusion step. Thus, the Applicant carried out a process wherein the co-extrusion liquid phase containing gellan was performed in absence of calcium salts, and observed that, surprisingly, the resulting capsules had the required spherical or substantially spherical shape and homogeneous sizes. However, the capsules thus obtained could not be used as such, because the shell was too soft and the resulting capsules were not breakable capsules; the Applicant found a solution to this subsequent technical problem by contacting the capsules with divalent metal ions, preferably calcium or magnesium ions, or by using organic acid solution, once the co-extrusion process is finished, and this finally lead to satisfactory breakable capsules.

Thus, this invention relates to a process for manufacturing seamless breakable capsules and to new seamless breakable capsules.

The process of the invention comprises a step (A) of co-extrusion of an external and hydrophilic liquid phase and an internal and lipophilic liquid phase, in order to form a capsule having a core comprising the internal and lipophilic phase and a shell comprising the external and hydrophilic phase; and a step (B) of washing and immersing the capsules into an aqueous solution preferably containing a curing agent, the curing agent being one of the means for making the shell breakable as required for the intended use; optionally a step (C) of drying the obtained capsules or optionally a step (D) of suspending the capsules into an fluid medium.

The co-extrusion process comprises three main stages: compound drop formation, shell solidification and capsule collection. The compound drop is a sphere of the liquid fill phase inside the shell phase. The liquid fill phase is hereinafter referred to as “the core”. The shell phase is hereinafter referred to as “the shell”.

According to the invention, the external liquid phase includes a gelling agent comprising gellan gum, alone or in combination with at least one suitable gelling agent, a filler, and a metal sequestering agent, the liquid preferably being aqueous, more preferably the liquid is water, preferably desionized or osmozed water.

By “gelling agent” in the meaning of this invention, it is referred to an agent able to convert an aqueous phase from a flowable or fluid liquid to a solid or a gel.

By “sequestering agent” in the meaning of this invention it is referred to any agent complexing, chelating or sequestering bivalent ions such as calcium or magnesium ions.

The term “substantially”, when referring to a number or value, means + or −10% of the value; when referring to a sphere, it means a distorted sphere which larger diameter is + or −10% of the diameter of the expected sphere.

The term “wet capsule” in the meaning of this invention, refers to a capsule which shell includes a positive amount of water. The term wet capsule is used for the calculation of percentages of ingredients in the final product or shell, as opposed to the calculation based on the dry weight of said final product or shell.

The breakable capsule according to the invention preferably has a crush strength from 0.01 to 5 kp, preferably from 0.1 to 2.5 kp, edge values being included. The crush strength of the capsule is measured by continuously applying a load vertically onto one particle until rupture. The crush strength of the capsules in the present invention is measured by using a texturometer TA.XT plus from Micro Stable System in compression mode or a LLOYD—CHATILLON Digital Force Gauge, Model DFIS 50, having a capacity of 25 Kg, a resolution of 0.02 Kg, and an accuracy of +/−0.15%. The force gauge is attached to a stand; the capsule is positioned in the middle of a plate that is moved up with a manual thread screw device. Pressure is then applied manually and the gauge records the maximum force applied at the very moment of the rupture of the capsule, (measured in Kg or in Lb). Rupture of the capsule results in the release of the core.

Gellan gum is a hydrocolloid which, according to the invention, can be used as the sole gelling agent of the external liquid phase, or in combination with at least one other gelling agent. Other suitable gelling agents may be alginates, agar, carragheenan, pectines, xanthan gum, Arabic gum, tara gum, ghatti gum, karaya gum, dextran, curdlan, welan gum, rhamsan gum or modified starches. Suitable gellan gums are for example, but not limited to deacylated gellan gum. Kelcogel® can be mentioned as a suitable gellan gum.

The amount of gelling agent present in the shell is 4 to 95%, preferably 5 to 75%, even more preferably is 10 to 50%, more preferably 12 to 40% by weight of the total dry weight of the shell.

When used in combination with at least another gelling agent, the weight ratio between gellan gum and the other gelling agent(s) is from 80/20 to 20/80, preferably 75/25 to 25/75, and even more preferably from 60/40 to 50/50.

Preferably, the weight ratio of gelling agent/dried shell is greater than 10%, preferably greater than 12, more preferably greater than 15%.

The filler is any suitable material that can increase the percentage of dry material in the external liquid phase or bring filming properties. Increasing the dry material amount in a shell results in solidifying the shell, and in making it physically more resistant or impermeable. Preferably, the filler is selected from the group comprising starch derivatives such as dextrin, maltodextrin, polyol, cyclodextrin (alpha, beta or gamma), or cellulose derivatives such as hydroxypropylmethylcellulose (HPMC), hydroxypropylcellulose (HPC), methylcellulose (MC), carboxymethylcellulose (CMC), polyethylene glycol derivatives, polyvinyl alcohol, polyols or mixture thereof.

The amount of filler in the shell is at most 98.5%, preferably from 25 to 95% and even more preferably from 50 to 80% by weight on the total dry weight of the shell.

Using a divalent metal sequestering or complexing agent allows trapping the divalent metal ions which are possibly present in the components of the liquid phase including water and which have a gelling effect on gellan. Thus, the use of a divalent metal sequestering agent, preferably of a calcium ion sequestering agent, allows the gellan to be co-extruded without undesirable or uncontrollable gelling during the co-extrusion.

The amount of sequestering agent is at most 2%, preferably at most 1% and even more preferably at most 0.5% by weight of the total dry weight of the shell.

Preferably, the water used for the external phase is deionized water and/or osmozed water; using processing water remains possible but needs adjusting the amount of divalent metal sequestering agent.

The sequestering agent is a metal salt, preferably selected from the group comprising trisodium citrate, trisodium phosphate, tetrasodium pyrophosphate, sodium hexametaphosphate and mixtures thereof.

The hydrophilic external liquid phase may further comprise at least one plasticizer, which may be at least one of glycerol, sorbitol, maltitol, triacetine or polyethylene glycol type product, or a polyalcohol with plasticizing or humectant properties. Advantageously, the hydrophilic external liquid phase further comprises at least one colouring agent or pigment; according to a first embodiment, the colouring agent or the pigment is in a form of a powder or a suspension stable in an aqueous medium. According to another embodiment of the invention, the liquid phase may include perfumes, aromas, fragrances or any odoring agent.

According to one embodiment of the invention, the co-extrusion step (A) of the process can be performed at a temperature being from room temperature to 100° C. Advantageously, it is performed at room temperature, which means between 18 and 30° C., preferably 20-25° C. under atmospheric pressure.

The co-extrusion step is a synchronous extrusion of two liquids: the external and hydrophilic liquid phase, and the internal and lipophilic liquid phase which can be performed using an apparatus and a process as described in EP 513603, the disclosure of which is herein incorporated by reference.

According to an embodiment of the invention, after the co-extrusion step (A), the solidification step is performed by keeping cold the capsules in order to ensure correct gelling of the shell, for example by contacting them with a cold bath. The cold bath may preferably be cold oil or cold emulsion. Cold means any temperature below 18^{-°} C., preferably the temperature is from 2 to 10° C., more preferably 4 to 6° C.

According to an embodiment of the invention, the capsules may then be centrifuged in order to remove the surplus oil, and/or washed with organic solvent (such as acetone, ethyl acetate, ethanol, petroleum ether, etc.) also to remove the surplus oil, and optionally dried in a air flow at controlled temperature and humidity. The relative humidity of the drying air is 20% to 60%, preferably 30 to 50%; the temperature of the drying air is of 15 to 60° C., preferably 35 to 45° C.

According to another embodiment, the capsules are preferably immersed into an aqueous solution or an emulsion containing a curing agent which comprises a divalent salt and optionally an acid. The effect of the immersion step is to wash out the oil remaining at the periphery of the capsule, and to gradually strengthen the shell, notably through dehydration and osmotic equilibrium.

According to one embodiment of the invention, after immersion, the capsules are dried in the same conditions as mentioned above. According to another embodiment of the invention, after immersion, the capsules are not dried.

The curing agent preferably comprises divalent metal ions, or a mixture of divalent metal ions, such as calcium ions or magnesium ions.

The aqueous solution or emulsion containing the curing agent is preferably a divalent metal salt solution, preferably containing calcium or magnesium salts, more preferably, calcium dichloride, calcium carbonate, calcium sulfate or dicalcium phosphate. This solution may be the aqueous phase of an oil-in-water emulsion. This solution can be at a temperature comprised between 2° C. and room temperature. Advantageously, the aqueous solution containing the curing agent is maintained under acid conditions of pH, and preferably at a pH less than 5, more preferably from 2 to 4. According to a preferred embodiment of the invention, the aqueous solution or emulsion containing a curing agent is a calcium chloride solution having a pH of 3 to 4.

The aqueous solution containing the curing agent can also contain preservatives or bactericides such as benzoate, parabens, diols, cetylpyridinium chloride, diazolidinyl urea or any preservatives used for food, pharmaceutical or cosmetic products.

According to one embodiment of the invention, the process comprises the steps of co-extruding the above mentioned external and internal liquid phases, optionally solidifying and/or gelling the surface of the shell by keeping the capsule under cold conditions, as explained herein above, optionally centrifugating, optionally washing the so-obtained capsules with an organic solvent, immersing the resulting capsules into an aqueous solution containing a curing agent, and optionally drying the capsules.

According to one embodiment of the invention, the solidifying/gelling/curing steps can be gathered into a single step, for example by dipping the capsules into a bath, under cold conditions, containing the divalent metal salts, preferably calcium or magnesium salts, more preferably, calcium dichloride, calcium sulfate or dicalcium phosphate. This bath may be an oil-in-water emulsion.

The capsules manufactured through the process according to the invention are substantially or perfectly spherical and very homogeneous in size.

This invention also relates to breakable capsules which are preferably seamless capsules susceptible to be obtained through the process according to the invention.

The capsule of the invention comprises a core and a shell, and said shell includes a gelling agent comprising gellan gum alone or in combination with another gelling agent, a filler, and a divalent metal sequestering agent.

Preferably the gelling agent of the shell is a combination of gellan and of at least one other gelling agent selected from the group consisting of gelatin and hydrocolloids such as agar, carragheenan, pectins, xanthan gum, alginate, tara gum, arabic gum, ghatti gum, caroub gum, cellulose gum, dextran, curdlan, welan gum, rhamsan gum or modified starches.

According to a preferred embodiment of the invention the filler and the sequestering agent, are as described hereinabove.

According to another embodiment, the shell further comprises a plasticizer as described hereinabove.

The amount of plasticizer ranges from 0.1% to 30% by weight, preferably from 2% to 15% by weight, and even more preferably from 3 to 10% by weight of the total dry weight of the shell.

According to the intended use of said capsules, the shell may contain other additives such as perfumes, aromas, or any flavoring agent.

According to the intended use of said capsule, the shell may comprise coloring agent such as pigments, titanium dioxide, iron oxides, carbon black, or any type of food, oral care, cosmetic or pharmaceutical pigment such as Covasorb colors distributed by LCW.

The shell of a breakable capsule according to the invention represents by weight 8 to 50% of the total weight of said capsule, preferably 10 to 40%, more preferably 20 to 30%.

The amount of water present in the shell is of 1 to 60%, preferably 5 to 40% the capsule remaining breakable even at the higher percentages.

According to a preferred embodiment, the breakable capsule according to the invention has a crush strength of from 0.01 to 5, preferably from 0.01 to 2.5 kp.

Advantageously, the shell thickness of the capsule is 10-500 microns, preferably 30-150 microns, more preferably 50-60 microns. The ratio diameter of the capsule/thickness of the shell is in the range of 1 to 100, preferably 5 to 30.

The core of the capsule is preferentially composed of a mixture of materials or products which are lipophilic or partially soluble in ethanol, or of molecules formulated as oil/water/oil emulsions.

The core of a breakable capsule according to the invention represents by weight 50 to 92% of the total weight of said capsule, preferably 60 to 90%, more preferably 70 to 80%.

The core of the capsule may be composed of one or more lipophilic solvents conventionally used in the food, pharmaceutical or cosmetic industries. In a preferred embodiment, these lipophilic solvents may be triglycerides, especially medium chain triglycerides, and in particular triglycerides of caprylic and capric acid, or mixtures of triglycerides such as vegetable oil, hydrogenated oil, coconut oil, palm oil, olive oil, sunflower oil, corn oil, linseed oil, cottonseed oil, groundnut oil, grape seed oil, wheat germ oil, fish oil, beet fat, mineral oils and silicone oils. The amount of lipophilic solvent in the core of a capsule according to the invention is of the order of 0.01 to 90%, preferentially 25 to 75%, of the total weight of the capsule.

The core may also comprise one or more aromatic or fragrance molecules as conventionally used in the formulation of flavoring or fragrance compositions. Mention will in particular be made of aromatic, terpenic and/or sesquiterpenic hydrocarbons, and more particularly essential oils, alcohols, aldehydes, phenols, carboxylic acids in their various forms, aromatic acetals and ethers, nitrogenous heterocycles, ketones, sulfides, disulfides and mercaptans which may be aromatic or non aromatic. It may also comprise one or more molecules or extracts for cosmetic use.

The core may also comprise one or more fillers as used in aromatic emulsions. Mention will be made of dammar gum, wood resins of the ester gum type, sucrose acetate isobutyrate (SAIB) or brominated vegetable oils. The function of these weighting agents is to adjust the density of the liquid core.

The core may also comprise one or more sweeteners, which may be provided in the form of a solution or suspension in ethanol. Examples of suitable sweeteners may be, but is not limited to, aspartame, saccharine, NHDC, sucralose, acesulfame, neotame, thaumatin, steviosides, etc.

The core may also comprise one or more “sensate” aromatic agents, which provide either a freshening effect or a hot effect in the mouth. Suitable freshening agents may be, but are not limited to, menthyl succinate and derivatives thereof, in particular Physcool® marketed by the Applicant. A suitable hot effect agent may be, but is not limited to, vanillyl ethyl ether.

The flavoring agents that can be solubilized in the solvent of the core of the capsule include, but are not limited to, natural or synthetic aromas and/or fragrances. Examples of suitable fragrances are fruity, confectionery, floral, sweet, woody fragrances. Examples of suitable aromas are vanilla, coffee, chocolate, cinnamon, mint. The core may also comprise a lipophilic color such as fake colors but also natural colors such as paprika oleoresin, turmeric oleoresin, carotenes, chlorophyllin, or any other suitable natural coloring product. The core may also include lipophilic active agents, such as vitamins, more preferably vitamins B; fatty acids, preferably omega 3 and natural extracts of plants.

The capsules according to the invention can be included in various products, such as food products, oral care products, nutraceutical products, pharmaceutical products, cleaning products and cosmetic products. The invention thus relates to a food product including breakable capsules according to the invention; an oral care product including breakable capsules according to the invention, preferably a toothpaste including breakable capsules according to the invention; a pharmaceutical product including breakable capsules according to the invention; a fragrance including breakable capsules according to the invention.

The capsules of the invention may be within a slurry, in suspension in a gel, preferably carried out with a gel forming agent such as xanthan gum, gellan gum, CMC or Carbopol, araboxymethyl cellulose, or any polymer commonly used as suspending agent and optionally comprising preservatives and stabilizers.

The total weight of the capsule of the invention depends on its diameter and on the amount of core filling the shell. According to an embodiment of the invention, the total weight of the capsule is within the range of 0.1 to 50 mg, preferably 0.2 to 20 mg, more preferably 0.5 to 10 mg.

The invention is hereunder illustrated by the following examples, which should not be considered as limiting the scope of the invention.

### EXAMPLES

### Example 1

Menthol Capsules (referred as 3039/A1) are prepared by co-extruding an outer liquid phase and an internal liquid phase presenting the following compositions:

| Outer liquid phase | %/total | | % wet |
|---|---|---|---|
| Dry matter: 15.0% | weight | %/dry matter | capsule |
| gellan | 2.000% | 13.33 | 1.482 |
| Sorbitol | 1.000% | 6.67 | 0.741 |
| Dextrin Cristal Tex | 11.400% | 76.00 | 8.445 |
| 648 | | | |
| Sodium citrate | 0.200% | 1.33 | 0.148 |
| Citric acid | 0.1% | 0.67 | 0.074 |
| unipure blue | 0.300% | 2.00 | 0.222 |
| pigment CI77007 | | | |
| Deionized water | 85.000% | | 62.968 |
| | 100.000% | 100 | |
| Internal liquid | | | |
| phase | % | % | |
| Ethanol | 5.0000% | 5% | |
| Miglyol 812S | 81.5000% | 81.5% | 22.245% |
| Menthol codex | 13.5000% | 13.5% | 3.685% |
| | 100.0000% | 100.00% | 100% |

The obtained capsules are separated into two batches referred as A1a and A1b. Capsules from each batch are cooled at 4° C. for 1 h, washed with desionised water and then immersed in a bath containing an aqueous solution of calcium chloride (0.1% for A1a and 1% for A1b) at pH=3.5 at T=20° C. during 15 minutes.

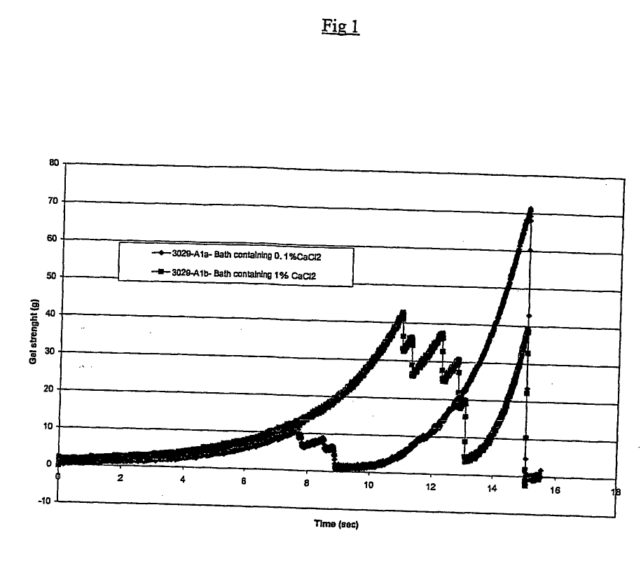

Wet capsule crush strength (gel strength) is then measured for both capsules Ala and Alb using a texturometer TA.XT plus from Micro Stable System to compare influence of concentration of calcium (the results are presented on FIG. 1).

Wet capsule strength is higher using 1% CaCl2 solution than using 0.1% CaCl2 solution.

After drying, crush strength of the capsules is measured using a texturometer in compression mode.

| | 3039/A1a | 3039/A1b |
|---|---|---|
| Crush strength | 184 g | 186.6 g |
| (dry capsules) | | |

The obtained capsules present the following physical 20 characteristics:
diameter: 2 mm,
thickness of the shell: 0.096 mm,
total weight: 4 mg,
weight of the core: 2.8 mg (70%),
weight of the shell: 1.2 mg (30%).

Such capsules are then placed into a clear toothgel and bring nice visual effect of spherical blue capsules liberating menthol when broken.

### Example 2

Cinnamon Capsules (referenced as 4053/F1) are prepared by co-extruding an outer liquid phase and an internal liquid phase presenting the following compositions:

| Outer liquid phase | %/total | %/dry |
|---|---|---|
| Dry matter: 13.0% | weight | matter |
| gellan | 2.000% | 15.38% |
| Sorbitol | 1.900% | 14.62% |
| Dextrin Cristal Tex 648 | 8.500% | 65.38% |
| Sodium citrate | 0.200% | 1.54% |
| Calcium citrate | 0.100% | 0.77% |
| Titanium dioxide | 0.300% | 2.31% |
| Osmosed water | 87.000% | 100% |
| | 100.000% | |
| Internal liquid | %/total | % without |
| phase | weight | ethanol |
| Ethanol | 5.0000% | |
| Miglyol 812S | 58.9000% | 85.79% |
| Cinnamon | 19.6000% | 14.21% |
| Physcool | 10.0000% | 10.53% |
| N-ethyl-p-menthane- | 6.5000% | 6.84% |
| 3-carboxamide | | |
| commercialy | | |
| available as WS3 | | |
| Total | 100.0000% | 100.00% |

The obtained capsules are cooled at 4° C. for 1 h, washed with deionised water and then immersed in a bath containing an aqueous solution containing 1.25% of calcium chloride at pH=3 at T=20° C. during 30 minutes.

The obtained capsules present the following physical characteristics:
diameter: 1.2 mm,
thickness of the shell: 0.053 mm,
total weight: 0.87 mg,
weight of the core: 0.62 mg (71.98%),
weight of the shell: 0.24 mg (28.02%),

Capsules are then incorporated into a toothpaste base containing mint flavour and cinnamon capsules 4053/F1 at a 0.2% use level. During brushing, cinnamon flavour is clearly identified showing good breakability of the capsules.

### Example 3

orange capsules (referred as 5053/C1) are prepared by coextruding an outer liquid phase and an internal liquid phase presenting the following compositions:

| Outer liquid phase | %/total | %/wet |
|---|---|---|
| Dry matter: 15.0% | weight | capsule |
| gellan | 2.000% | 0.95% |
| Sorbitol | 1.000% | 0.45% |
| Dextrin Cristal Tex | 11.4% | 5.36% |
| 648 | | |
| Sodium citrate | 0.200% | 0.01% |
| water | 84.5% | 40% |
| | 100.000% | |
| Internal liquid | | |
| phase | % | % |
| Orange flavour | 19.905% | 5.47% |
| Coconut oil | 80% | 47.7% |
| Paprika color | 0.095% | 0.06% |
| Total | 100.0000% | 100.00% |

Wet capsule crush strength (gel strength) is then measured using a texturometer TA.XT plus from Micro Stable System. Crushstrength value obtained is 15 g and these capsules are easily broken under the teeth.

The obtained capsules present the following physical characteristics:

| | | |
|---|---|---|
| Diameter: | 2.5 | mm |
| Thickness of the shell: | 0.32 | mm |
| Total weight: | 8.2 | mg |

Capsules are then placed into a suspension of xanthan gum to be applied to beverage application. Capsules can be swallowed or broken under the teeth to liberate the flavour into the mouth.

### Example 4

Menthol capsules (referred as 5025/B1) are prepared by coextruding an outer liquid phase and an internal liquid phase presenting the following composition:

| | | | | | % wet capsule | |
|---|---|---|---|---|---|---|
| | | | | | treated with acid | |
| | | | % wet capsule | | as calcium | |
| | % dry capsule | | untreated | | releasing agent | |
| | % | %/total | % | %/total | % | %/total |
| | shell | weight | shell | weight | shell | weight |
| Outer liquid | | | | | | |
| phase | | | | | | |
| gellan | 13.333% | 3.6% | 2% | 1.423% | 2% | 1.423% |
| Sorbitol | 6.667% | 1.8% | 1% | 0.712% | 1% | 0.712% |
| Dextrin | 76% | 20.52% | 11.4% | 8.111% | 11.4% | 8.111% |
| Cristal Tex | | | | | | |
| 648 | | | | | | |
| Sodium | 1.333% | 0.360% | 0.2% | 0.142% | 0.2% | 0.142% |
| citrate | | | | | | |
| Citric acid | 0.667% | 0.180% | 0.1% | 0.071% | 0.1% | 0.071% |
| Unipure blue | 2% | 0.54% | 0.3% | 0.213% | 0.3% | 0.213% |
| pigment | | | | | | |
| CI77007 | | | | | | |
| water | 0% | 0% | 85% | 60.480% | 85% | 60.480% |
| Internal | | | | | | |
| liquid phase | | | | | | |
| ethanol | 0% | 0% | 0% | 0% | 0% | 0% |
| Miglyol 812S | 47.368% | 34.579% | 47.368% | 13.664% | 47.368% | 13.664% |
| Menthol codex | 52.632% | 38.421% | 52.632% | 15.183% | 52.632% | 15.183% |
| Total | 100% | 73% | 100% | 28.8% | 100% | 28.8% |
| Total | | 100% | | 100% | | 100% |
| Crush | 94.31 g | | 5.09 g | | 15.09 g | |
| strength | | | | | | |

The treatment of wet capsules with an acid as calcium releasing agent allow the enhancing of the crush strength of the capsules.

1. A seamless breakable capsule comprising a core and a shell, wherein the shell includes a gelling agent comprising gellan gum alone or in combination with another gelling agent, a filler, and a divalent metal sequestering agent.

2. The seamless breakable capsule according to claim 1, wherein the gelling agent is a combination of gellan and one gelling agent selected from the group consisting of gelatin, agar, carrageenan, pectins, xanthan gum, cellulose gum, alginate, dextran, curdlan, welan gum, rhamsan gum or modified starches.

3. The seamless breakable capsule according to claim 1, wherein the amount of gelling agent present in the shell is 4 to 95%, preferably 5 to 75%, even more preferably is 10 to 50% by weight of the total dry weight of the shell.

4. The seamless breakable capsule according to claim 3, wherein the gelling agent is gellan gum alone.

5. The seamless breakable capsule according to claim 1, wherein, when used in combination with at least another gelling agent, the weight ratio between gellan gum and the other gelling agent (s) is from 80/20 to 20/80, preferably 75/25 to 25/75, and even more preferably from 60/40 to 50/50.

6. The seamless breakable capsule according to claim 1, wherein the filler is a starch derivative such as dextrin, maltodextrin, cyclodextrin and/or a cellulose derivative such as hydroxypropylmethylcellulose (HPMC), hydroxypropylcellulose (HPC), methylcellulose (MC), polyvinyl alcohols, polyols or mixtures thereof.

7. The seamless breakable capsule according to claim 1, wherein the amount of the filler in the shell is at most 98.5%, preferably from 25 to 95% and even more preferably from 50 to 80% by weight on the total dry weight of the shell.

8. The seamless breakable capsule according to claim 1, wherein the sequestering agent is a metal salt, preferably selected from the group consisting of trisodium citrate, trisodium phosphate, tetrasodium pyrophosphate, sodium hexametaphosphate and mixtures thereof.

9. The seamless breakable capsule according to claim 1, wherein the amount of sequestering agent is at most 2%, preferably at most 1% and even more preferably at most 0.5% by weight of the total dry weight of the shell.

10. The seamless breakable capsule according claim 1, wherein the shell further comprises an acid salt selected from the group comprising such as citrate, glucuronate, adipate, fumarate, gluconate and salt of glucono-delta-lactone, and mixtures thereof.

11. The seamless breakable capsule according to claim 1, having an crush strength from 0.01 to 5 kp.

12. The seamless breakable capsule according to claim 1, wherein the amount of plasticizer ranges from 0.1 to 30% by weight, preferably from 2% to 15% by weight and even more preferably from 3 to 10% by weight of the total dry weight of the shell.

13. The seamless breakable capsule according to claim 1, having an crush strength from 0.01 to 5 kp.

14. A process for manufacturing a seamless breakable capsule, comprising
co-extruding an external and hydrophilic liquid phase, and an internal and lipophilic liquid phase, in order to form a capsule constituted of a core comprising the internal and lipophilic phase, and a shell comprising the external and hydrophilic phase,
immersing into an aqueous solution containing a curing agent, wherein the external liquid phase includes a gelling agent comprising gellan gum alone or in combination with another gelling agent, a filler, and a divalent metal sequestering agent

15. The process for manufacturing a seamless breakable capsule according to claim 14, comprising:
co-extruding an external and hydrophilic liquid phase, and an internal and lipophilic liquid phase, in order to form a capsule constituted of a core comprising the internal and lipophilic phase and a shell comprising the external and hydrophilic phase, optionally solidifying and/or gelling the surface of the shell by keeping the capsule under cold conditions,
optionally washing the so-obtained capsule with an organic solvent,
immersing into an aqueous solution containing a curing agent.
optionally drying the capsule.

16. The process for manufacturing a seamless breakable capsule according to claim 14, wherein the curing agent comprises divalent ions, preferably calcium ions.

17. The process for manufacturing a seamless breakable capsule according to claim 14, wherein the aqueous solution containing a curing agent is a calcium chloride solution, which pH is preferably of 3 to 4.

18. The process for manufacturing a seamless breakable capsule according to claim 14, wherein the gelling agent is a combination of gellan and at least one other gelling agent selected from the group consisting of gelatin and hydrocolloids such as agar, carragheenan, pectins, xanthan gum, cellulose gum, alginate, dextran, curdlan, welan gum, rhamsan gum or modified starches, and mixtures thereof.

19. The process for manufacturing a seamless breakable capsule according to claim 14, wherein the filler is a starch derivative such as dextrin, maltodextrin, cyclodextrin, a cellulose derivative such as HPMC, HPC, MC and mixtures thereof.

20. The process for manufacturing a seamless breakable capsule according to claim 14, wherein the sequestering agent is a metal salt, preferably selected from the group comprising sodium carbonate, trisodium citrate, trisodium phosphate, tetrasodium pyrophosphate, sodium hexametaphosphate and mixtures thereof.

21. The process for manufacturing a seamless breakable capsule according to claim 14, wherein the external hydrophilic liquid phase further comprises a plasticizer, preferably selected from the group consisting of glycerol, sorbitol, maltitol, triacetine or PEG type, or another polyol with plasticizing properties, and mixtures thereof.

22. Slurry containing breakable capsules according to claim 1, in suspension in a gel formed with a gel forming agent such CMC, xanthan gum, or Carbopol, and optionally comprising preservatives and stabilizers.

23. Food product including breakable capsules according to claim 1.

24. Oral care product including breakable capsules according to claim 1.

25. Pharmaceutical product including breakable capsules according to claim 1.

26. Fragrance including breakable capsules according to claim 1.
